(19) United States
(12) Patent Application Publication
(10) Pub. No.: US 2020/0324135 A1
(43) Pub. Date: Oct. 15, 2020

(54) Device and method for treating eight cancers with ultraviolet radiation

(71) Applicant: Keith Reed Greenbaum, Providence, RI (US)

(72) Inventor: Keith Reed Greenbaum, Providence, RI (US)

(21) Appl. No.: 16/501,431

(22) Filed: Apr. 15, 2019

(51) Int. Cl. A61N 5/06

(52) U.S. Cl. CPC A61N 5/0613; A61N 5/0603; A61N 2005/0611; A61N 2005/0604; A61N 2005/061; A61N 2005/0661

(57) ABSTRACT

This is a device and method for treating eight kinds of cancer with ultraviolet radiation. The cancers include ovarian, urinary (including bladder), melanoma, uterine, adult leukemia, lymphoma (Non-Hodgkin's), lung and prostate. The main claim is that people with these cancers treated with this device and method will live longer than people not treated in this way. The device gives off ultraviolet radiation whose energy level in the ultraviolet B range is three watts per square meter. The method entails exposure to the device for twenty minutes, three times a week on non-consecutive days. The idea that exposure to ultraviolet that is intense enough might increase the lifespan of people with these cancers comes from an analysis of US state cancer death rates. The more intense the ultraviolet reaching the ground, the fewer people die.

This patent application is for a device and method for treating eight kinds of cancer using ultraviolet radiation. For at least eight cancers, there is the same geographic pattern—north. The more north we travel, the more people die.

This insight came when writing the last two chapters of a “big data” project on US cancer death rates. I hope to publish the longer work as a book called “Cancer in America: The Risk of Living North” by Keith Reed Greenbaum. While I refer to this document here as “the book”, I decided not to incorporate it as part of the patent application believing that the information I present is sufficient to stand on its own.

What explains this north pattern of dying from cancer? While I raise two possibilities in the book, I believe the strongest one is a change in sunlight as we go north. As we move away from the equator, sunlight reaches the ground more on an angle which makes it less intense. This includes the ultraviolet portion of sunlight which weakens. In models for eight kinds of cancer, I show a statistical connection between the diminished intensity of ultraviolet reaching the ground (in the largest city of the state) and an increase in deaths from cancer in that state as we move north.

If the intensity of ultraviolet can account for a substantial portion of the differences in the number of people dying from various cancers from one state to the next, then perhaps dying from cancer might be due in part to a lack of exposure to ultraviolet that is intense enough. This possibility constitutes the evidence for the device and method below for treating certain cancers. Turning this connection around the other way, perhaps the artificial exposure to ultraviolet of the necessary intensity might be an effective treatment for cancer.

How am I the first to uncover this connection between the diminished intensity of ultraviolet and dying from cancer? I had the good fortune to make contact with an expert at NOAA who works on the UV Index, a daily prediction of the intensity of ultraviolet reaching the ground in major US cities. At my request, he changed the UV numbers from daily to yearly form. This is what worked for revealing this connection between ultraviolet and cancer. The direction, however, turned out to be in the opposite direction than expected. If more people died from cancer the weaker ultraviolet became, then maybe ultraviolet might work as a treatment for cancer.

As I stated above, this possible connection between ultraviolet and cancer is the result of a “big data” project which includes virtually every person who died of a given cancer in the United States over the course of a three-year period. I intend to construct the machines based on the Hawaii sun, the place that the statistical result identifies as having the most intense ultraviolet over the course of the year in the United States, which is, in turn, linked to the fewest cancer deaths, generally speaking. The actual measurements will be modified to fit even more closely with my interpretation of the statistical result.

In sum, variations in death rates from cancer might be connected with a specific portion of naturally occurring sunlight that is not strong enough. This new knowledge provides a powerful foundation for a novel treatment which has a reasonable chance of working. The goal is to significantly increase the lifespan of those dying from these cancers.

As a final part of this introductory section, I chose eight cancer death categories which should fall under the purview of this patent. While this northern pattern of cancer deaths applies to deaths from most kinds of cancers, for these eight kinds, I was able to construct individual models which show both a northern pattern as well as the possibility that this pattern is the result of a connection with variations in the intensity of ultraviolet. I designate the following as the eight northern cancers: lung, melanoma, ovarian, urinary (including bladder), uterine, Non-Hodgkin's lymphoma, adult leukemia and prostate.

Because these are death categories, they include the extra auxiliary locations of cancers in that category. I specify such locations for two of the cancers as follows. The lung category includes “[m]alignant neoplasms of trachea, bronchus and lung”. Urinary tract cancer includes bladder cancer (some 54 percent) as well as cancers of the kidney, ureter and urethra.

Based on my calculations, the above cancers constitute close to half of all cancer deaths in the United States, some 49 percent (of the six hundred thousand and fifteen thousand expected to die during the year 2017, according to the American Cancer Society). This new device and method, then, has the potential to prolong the lives of around three hundred thousand people dying from cancer every year.

The key feature of the device is the intensity of ultraviolet which comes from the statistical result showing a link between stronger ultraviolet and fewer cancer deaths in the United States. This would be set to mimic the yearly average for ultraviolet intensity in the place with the strongest sun—Honolulu Hi. Based on measures of the yearly UVI (UV Index) for American cities, over a total of ten years (one plus nine) and modified by actual sun measurements on location, this proposed setting would maximize the chances for creating a cancer treatment that is both safe and effective.

The radiation would be almost entirely in the ultraviolet range. It would begin at the beginning of the higher energy UVB portion at a value of 280 nanometers. It would continue up through the end of the UV range ending with the weakest wavelength of the UVA range at 400 nanometers.

The key attribute of ultraviolet intensity is the B portion as measured by a Solarmeter Model 6.0 Standard UVB Meter (after milliwatts per square centimeter is converted to watts per square meter) which would be set at 3 watts per square meter. The B portion is foremost because the UV Index itself is a weighted measure which augments the role of the stronger B portion to focus on the effect of sunlight on human skin. This setting is based on an average of actual summer and winter measurements in Hawaii (near Waikiki Beach in Honolulu), this average itself being an attempt to mimic the year-round strength of ultraviolet in this version of the UV Index used in the research. This setting would maximize the chances of creating a cancer treatment that, while effective, might also be safer because the strength comes from a range that is an average of naturally occurring intensity values. The UV Index value would be between the lower actual measured average value of 6.6 and the UV Index value of 10, toward the high in the statistical research.

As the goal would be the minimum exposure necessary to achieve a therapeutic result, the device would include three sizes with the smallest effective version to be preferred. The two smaller versions would make shipping easier. The first would be a facial-style tanner with dimensions 22 inches wide, 9 inches deep and 14 inches tall. The Medium machine would have dimensions of about 2 feet tall and 1 foot wide. Both smaller machines would have four florescent-style tubes created with the ultraviolet specifications mentioned above. The large machine would be one row of five or six foot long florescent-style tubes preferably in a frame that can be tilted for use standing or lying. There would be a minimum of six such tubes in the row.

For all three size machines, the distance from the patient would be determined by the value of UVB to be set at 3 watts per square meter. The Small unit would be employed on the face but might also be employed in other areas related to the location of the cancer. The same is true for the Medium unit. For the Large machine, the patient would need to be undressed but covering the eyes with UV protective goggles, necessary as well for the two smaller machines.

While treatment time would be determined experimentally, the beginning goal would be 20 minutes per day three times a week. For the Large, full body version of the device, this would mean 10 minutes on the front and 10 minutes on the back. The time of a session would be worked up to this maximum gradually to prevent skin burning.

The first distinctive feature of these machines and this method is the purpose. This is probably the first ultraviolet-based device and method designed specifically for treating various cancers.

Second is its exclusive focus on only one of the three portions of sunlight. While the energy from sunlight is typically divided (from highest energy) into ultraviolet, visible light and infrared (heat), this device and method is careful to employ only one of the three portions—ultraviolet. Great care will be exercised to ensure that no energy from the infrared portion is included. As for visible light, this too would be excluded with the exception of a small portion to make sure that the human eye can discern when the unit is on.

Third, it is the only device specifically designed to mimic the intensity of ultraviolet in the major American city with the highest yearly value. Of course, the purpose here is to imitate conditions based on the statistical connection between the intensity of ultraviolet reaching the ground in that place over the year and fewest deaths from cancer of various types.

For the eight premiere northern cancers, there are two grouping. In the first group of five—lung, melanoma, ovarian, urinary and uterine, the individual models point to diminished ultraviolet in sunlight as we travel north as the strongest explanation. In the second grouping, including lymphoma, leukemia and prostate, the strongest result in the book pointed to a different environmental factor that varies as we travel north—the Earth's stronger magnetic field.

Based on the hunch that this same northern pattern might be the result of one rather than two environmental factors, I reworked this second group to fit the possibility that these too are linked with the diminished strength of “sunlight”. In the final two chapters of the book, I did this by creating new models after the three cancers were added together. Here for this application, I went further by redoing the final individual models to see if I could replace Earth magnetism with ultraviolet. This was extremely easy to do for lymphoma and adult leukemia.

For prostate, the exercise yielded a weaker result with a statistical significance value of 0.009, under 0.01, my cut-off, surely better than the standard 0.05 cut-off, but still, not as strong as the other results in terms of reliability. I decided to include prostate here because the thrust of the evidence—considering both individual models as well as the aggregated models at the end of the book—led me to believe that prostate is also a northern cancer. For this reason, it deserves to be with this group of the eight northern cancers and I believe that there is a reasonable chance that the above treatment might work for this cancer as well.

Now I present more detail about the evidence which suggests a link between deaths from these various northern cancers and diminished ultraviolet in sunlight. I start with the yearly death rates for each cancer by state for a given set of relatively recent years. This includes all of the deaths from a given cancer during the time period as best as we were able to record. I believe that all of these death rate lists were from the CDC web site (The Center for Disease Control) using a table called Health Data Interactive, a project that close in July, 2016. Using a data set of state-level items I developed over a twenty-year period, most from public sources, I tested many items in a multiple regression model to see what might be linked with the pattern of deaths from that cancer. In the final model, I showed a link between one environmental item that varies in a northern pattern and dying from that cancer.

Lastly, using the regression equation, I created a new “predicted” death rate list for the cancer by holding constant the less interesting items (by inserting their mean value in the equation) and letting vary only the northern item. In this new death rate list, the difference between the state with the highest death rate and the one with the lowest death rate was the size decrease in deaths that was due exclusively to this environmental item that changed as we moved from south to north. For the version in this application, all of these new death rate constructions vary only ultraviolet based on the yearly intensity of ultraviolet in the largest city of the state.

I soon present these percent decreases in death rates as one way to represent the power of the finding, the possibility that deaths from each cancer might have dropped substantially if ultraviolet had been stronger in intensity. This provides the evidence for the size of this possible connection and the foundation for the new treatment for cancer implicit in this device.

For all eight cancer death models, I show a link between ultraviolet and death rates using one of two versions of the UV Index, the first from 1996 in Table 12-3 and the second from 2006-2014 in Table 7-3, (the out of order table numbers being from the book). (“District” means Washington D.C.).

TABLE 12-3
[ultra96] ultraviolet. Average annual UVI (UV Index) score for
the largest city in the state over the course of the year 1996.

| | |
|---|---|
| Alaska | 1.4 |
| Washing | 2.7 |
| Vermont | 2.9 |
| Oregon | 3.0 |
| Maine | 3.1 |
| North Da | 3.1 |
| New Hamp | 3.2 |
| Minnesot | 3.2 |
| Michigan | 3.3 |
| Wisconsi | 3.3 |
| Massachu | 3.3 |
| Ohio | 3.4 |
| Connecti | 3.4 |
| Rhode Is | 3.4 |
| Illinois | 3.5 |
| South Da | 3.5 |
| Montana | 3.5 |
| New York | 3.6 |
| Indiana | 3.7 |
| Iowa | 3.7 |
| Nebraska | 3.8 |
| Pennsylv | 3.8 |
| Maryland | 3.9 |
| Delaware | 3.9 |
| New Jers | 3.9 |
| Idaho | 4.0 |
| West Vir | 4.0 |
| District | 4.0 |
| Kentucky | 4.1 |
| Missouri | 4.1 |
| Virginia | 4.4 |
| North Ca | 4.6 |
| Utah | 4.7 |
| Wyoming | 4.7 |
| Kansas | 4.7 |
| Arkansas | 4.8 |
| Tennesse | 4.8 |
| Colorado | 4.9 |
| Georgia | 5.1 |
| Oklahoma | 5.2 |
| South Ca | 5.3 |
| Mississi | 5.5 |
| Nevada | 5.7 |
| Alabama | 5.8 |
| Texas | 5.9 |
| Californ | 5.9 |
| Louisian | 5.9 |
| New Mexi | 6.1 |
| Arizona | 6.2 |
| Florida | 6.9 |
| Hawaii | 9.6 |

TABLE 7-3
[uvi0614] UVI (UV Index) between 2006 and 2014.
Ultraviolet. Average annual UVI (UV Index) score over
a nine year period for the largest city in the state.

| | |
|---|---|
| Alaska | 1.9 |
| Washing | 3.5 |
| Oregon | 3.6 |
| Vermont | 3.6 |
| Minnesot | 3.9 |
| North Da | 3.9 |
| Maine | 3.9 |
| Michigan | 4.0 |
| New Hamp | 4.0 |
| Wisconsi | 4.1 |
| Illinois | 4.2 |
| Connecti | 4.2 |
| Massachu | 4.2 |
| Ohio | 4.2 |
| South Da | 4.2 |
| Rhode Is | 4.2 |
| Iowa | 4.3 |
| New York | 4.4 |
| Montana | 4.4 |
| Pennsylv | 4.4 |
| Indiana | 4.5 |
| Nebraska | 4.5 |
| Maryland | 4.6 |
| New Jers | 4.6 |
| District | 4.6 |
| Delaware | 4.6 |
| West Vir | 4.6 |
| Kentucky | 4.8 |
| Missouri | 4.8 |
| Idaho | 4.9 |
| Kansas | 5.2 |
| Virginia | 5.4 |
| North Ca | 5.4 |
| Wyoming | 5.5 |
| Tennesse | 5.5 |
| Arkansas | 5.6 |
| Oklahoma | 5.6 |
| Utah | 5.7 |
| Colorado | 5.8 |
| Georgia | 6.0 |
| South Ca | 6.3 |
| Nevada | 6.3 |
| Mississi | 6.4 |
| Texas | 6.9 |
| Arizona | 6.9 |
| Californ | 7.0 |
| New Mexi | 7.0 |
| Alabama | 7.0 |
| Louisian | 7.2 |
| Florida | 8.5 |
| Hawaii | 11.1 |

Both show the least intense ultraviolet in Alaska (Anchorage) and the most intense in Hawaii (Honolulu). I present these results suggesting that the death rate in Alaska could have been this percent lower had it had the same intensity of ultraviolet in sunlight that Hawaii had. These numbers for the eight northern cancers follow below in a little more detail.

The Eight Northern Cancers and their Link to the Insufficient Intensity of Ultraviolet

### Lung Cancer

I will start with lung which happens to be the biggest cancer in terms of the number of people who die from it, (some 27 percent of the total). Table 3-1 is the yearly death rate from lung cancer by state during the years 2011 and 2013.

TABLE 3-1
lung1113. Lung cancer death rate between 2011 and 2013,
yearly average (per hundred thousand people), by state.

| | |
|---|---|
| Utah | 22.5 |
| Colorado | 39.9 |
| Californ | 43.7 |
| New Mexi | 46.8 |
| District | 46.9 |
| Hawaii | 49.0 |
| Alaska | 49.1 |
| Texas | 50.4 |
| Wyoming | 52.0 |
| Idaho | 53.2 |
| Arizona | 55.8 |
| Minnesot | 57.2 |
| Washing | 58.2 |
| New Jers | 59.4 |
| Maryland | 59.6 |
| New York | 59.6 |
| Connecti | 60.3 |
| Georgia | 61.6 |
| North Da | 62.4 |
| Virginia | 62.7 |
| Nevada | 63.2 |
| Nebraska | 64.3 |
| Massachu | 64.9 |
| Montana | 66.8 |
| Illinois | 67.4 |
| Oregon | 67.7 |
| Wisconsi | 67.9 |
| Kansas | 68.8 |
| South Da | 69.3 |
| Vermont | 71.2 |
| New Hamp | 71.4 |
| North Ca | 73.7 |
| Iowa | 74.9 |
| Rhode Is | 75.6 |
| Pennsylv | 76.3 |
| Michigan | 76.5 |
| South Ca | 77.6 |
| Florida | 77.8 |
| Louisian | 78.1 |
| Delaware | 79.1 |
| Indiana | 80.0 |
| Ohio | 83.3 |
| Alabama | 84.7 |
| Missouri | 85.3 |
| Oklahoma | 85.4 |
| Mississi | 86.5 |
| Tennesse | 87.4 |
| Maine | 91.1 |
| Arkansas | 95.5 |
| West Vir | 100.2 |
| Kentucky | 103.4 |

The lung cancer model is in Table 3-4 consisting of two items: the percent who have attended some college (fewer deaths) and the UVI value (UV index score for the largest city of the state between 2006 and 2014). The higher the ultraviolet, the fewer the lung cancer deaths.

TABLE 3-4
The Lung Cancer Model: The Role of Not Strong Enough
Ultraviolet in Sunlight (more deaths).
Model Summary

| | | R | Adjusted R | Std. Error of the |
|---|---|---|---|---|
| Model | R | Square | Square | Estimate |
| 1 | .876(a) | .767 | .757 | 7.9234 |

(a)Predictors: (Constant), UVI0614, SUMCOLIG

Coefficients(a)

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | 174.371 | 8.954 | | 19.473 | .000 |
| | SUMCOLIG | −2.103 | .169 | −.869 | −12.419 | .000 |
| | UVI0614 | −2.126 | .756 | −.197 | −2.813 | .007 |

(a)Dependent Variable: LUNG1113

Correlations

| | | SUMCOLIG | UVI0614 | LUNG1113 |
|---|---|---|---|---|
| SUMCOLIG | Pearson | 1 | −.080 | −.853(**) |
| | Correlation | | | |
| | Sig. | . | .578 | .000 |
| | (2-tailed) | | | |
| | N | 51 | 51 | 51 |
| UVI0614 | Pearson | −.080 | 1 | −.128 |
| | Correlation | | | |
| | Sig. | .578 | . | .372 |
| | (2-tailed) | | | |
| | N | 51 | 51 | 51 |
| LUNG1113 | Pearson | −.853(**) | −.128 | 1 |
| | Correlation | | | |
| | Sig. | .000 | .372 | . |
| | (2-tailed) | | | |
| | N | 51 | 51 | 51 |

(**)Correlation is significant at the 0.01 level (2-tailed).

Holding constant the huge effect of college results in the new predicted death rate for lung cancer which shows the effect of ultraviolet.

This predicted death rate list appears in Table 3-5 showing the fewest deaths in Hawaii, 55.2 per hundred thousand people, and the most in Alaska with 74.8.

TABLE 3-5
lunguvil. lung - UV Index list. Best view of lung cancer death
rates by state after setting aside the huge effect of college
(fewer deaths). Predicted deaths (per hundred thousand) based
on actual death rates in 2011-2013 but varying only insufficient
ultraviolet reaching t ground (more deaths).

| | |
|---|---|
| Hawaii | 55.2 |
| Florida | 60.8 |
| Louisian | 63.5 |
| Alabama | 64.0 |
| New Mexi | 64.0 |
| Californ | 64.0 |
| Texas | 64.2 |
| Arizona | 64.2 |
| Mississi | 65.2 |
| South Ca | 65.5 |
| Nevada | 65.5 |
| Georgia | 66.1 |
| Colorado | 66.5 |
| Utah | 66.7 |
| Arkansas | 66.9 |
| Oklahoma | 66.9 |
| Tennesse | 67.2 |
| Wyoming | 67.2 |
| North Ca | 67.4 |
| Virginia | 67.4 |
| Kansas | 67.8 |
| Idaho | 68.4 |
| Kentucky | 68.6 |
| Missouri | 68.6 |
| West Vir | 69.1 |
| Delaware | 69.1 |
| New Jers | 69.1 |
| Maryland | 69.1 |
| District | 69.1 |
| Indiana | 69.3 |
| Nebraska | 69.3 |
| Pennsylv | 69.5 |
| New York | 69.5 |
| Montana | 69.5 |
| Iowa | 69.7 |
| Ohio | 69.9 |
| Rhode Is | 69.9 |
| Illinois | 69.9 |
| South Da | 69.9 |
| Connecti | 69.9 |
| Massachu | 69.9 |
| Wisconsi | 70.1 |
| Michigan | 70.3 |
| New Hamp | 70.3 |
| Maine | 70.6 |
| North Da | 70.6 |
| Minnesot | 70.6 |
| Vermont | 71.2 |
| Oregon | 71.2 |
| Washing | 71.4 |
| Alaska | 74.8 |

Using the highest death rate as the base: 74.8/1=55.2/x. 55.2=74.8x. x=55.2/74.8=0.738. With the highest level of ultraviolet, the death rate is only some 74 percent of the original size. 1−0.73796=0.26. Due to ultraviolet, the death rate from lung cancer in the place with the highest ultraviolet is 0.262 lower, or about 26 percent lower.

### Melanoma

Next is melanoma. Table 6-1 shows the death rate by state between 2008 and 2010.

TABLE 6-1
melanoma. Average yearly death rate from
melanoma between 2008 and 2010 by state.

| | |
|---|---|
| District | 1.4 |
| Hawaii | 2.3 |
| Alaska | 2.7 |
| Georgia | 3.0 |
| Texas | 3.1 |
| Mississi | 3.1 |
| New York | 3.1 |
| Louisian | 3.2 |
| Illinois | 3.3 |
| Californ | 3.4 |
| South Da | 3.4 |
| North Da | 3.4 |
| Nevada | 3.5 |
| Minnesot | 3.5 |
| Maryland | 3.6 |
| Michigan | 3.6 |
| South Ca | 3.7 |
| New Jers | 3.7 |
| Delaware | 3.8 |
| New Mexi | 3.9 |
| Rhode Is | 3.9 |
| Connecti | 3.9 |
| New Hamp | 3.9 |
| Arkansas | 4.0 |
| Virginia | 4.0 |
| Wisconsi | 4.0 |
| Utah | 4.1 |
| Washing | 4.1 |
| Alabama | 4.2 |
| Arizona | 4.2 |
| North Ca | 4.2 |
| Montana | 4.2 |
| Wyoming | 4.3 |
| Massachu | 4.3 |
| Tennesse | 4.4 |
| Kansas | 4.4 |
| Missouri | 4.5 |
| Indiana | 4.5 |
| Ohio | 4.5 |
| Colorado | 4.6 |
| Oklahoma | 4.6 |
| Kentucky | 4.6 |
| Pennsylv | 4.6 |
| Iowa | 4.6 |
| Florida | 4.7 |
| Idaho | 4.8 |
| Nebraska | 4.8 |
| Maine | 4.9 |
| Oregon | 4.9 |
| Vermont | 5.1 |
| West Vir | 5.3 |

The melanoma model appears in Table 6-5 with three items. Having a dog, more deaths, older age (65 or older), more deaths, and ultraviolet in 1996, fewer deaths.

TABLE 6-5
The Melanoma Model: The Role of Having a Dog (more deaths)
and not strong enough ultraviolet (more deaths).
Model Summary

| | | R | Adjusted R | Std. Error of the |
|---|---|---|---|---|
| Model | R | Square | Square | Estimate |
| 1 | .690(a) | .476 | .442 | .5537 |

(a)Predictors: (Constant), ULTRA96, AGE65IN6, DOGHOUSE

Coefficients(a)

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | .128 | .770 | | .166 | .869 |
| | DOGHOUSE | 6.253E−02 | .012 | .573 | 5.009 | .000 |
| | AGE65IN6 | .225 | .048 | .509 | 4.687 | .000 |
| | ULTRA96 | −.243 | .063 | −.437 | −3.866 | .000 |

(a)Dependent Variable: MELA810

Correlations

| | | DOGHOUSE | AGE65IN6 | ULTRA96 | MELA810 |
|---|---|---|---|---|---|
| DOGHOUSE | Pearson | 1 | −.175 | .326(*) | .3420 |
| | Correlation | | | | |
| | Sig. | . | .220 | .020 | .014 |
| | (2-tailed) | | | | |
| | N | 51 | 51 | 51 | 51 |
| AGE65IN6 | Pearson | −.175 | 1 | .084 | .372(**) |
| | Correlation | | | | |
| | Sig. | .220 | . | .560 | .007 |
| | (2-tailed) | | | | |
| | N | 51 | 51 | 51 | 51 |
| ULTRA96 | Pearson | .326(*) | .084 | 1 | −.208 |
| | Correlation | | | | |
| | Sig. | .020 | .560 | . | .144 |
| | (2-tailed) | | | | |
| | N | 51 | 51 | 51 | 51 |
| MELA810 | Pearson | .342(*) | .372(**) | −.208 | 1 |
| | Correlation | | | | |
| | Sig. | .014 | .007 | .144 | . |
| | (2-tailed) | | | | |
| | N | 51 | 51 | 51 | 51 |

(*)Correlation is significant at the 0.05 level (2-tailed).
(**)Correlation is significant at the 0.01 level (2-tailed).

Table 6-6 shows the new predicted death rate holding constant dog and older age to see the size of the effect of ultraviolet on death rates from melanoma.

TABLE 6-6
melasun. Predicted death rate from melanoma after setting aside
the effects of having a dog (more deaths) and older age (more
deaths) in order to see the possible effect of insufficiently
strong ultraviolet in sunlight on death rates (more deaths).

| | |
|---|---|
| Hawaii | 2.67 |
| Florida | 3.32 |
| Arizona | 3.49 |
| New Mexi | 3.52 |
| Californ | 3.57 |
| Louisian | 3.57 |
| Texas | 3.57 |
| Alabama | 3.59 |
| Nevada | 3.61 |
| Mississi | 3.66 |
| South Ca | 3.71 |
| Oklahoma | 3.74 |
| Georgia | 3.76 |
| Colorado | 3.81 |
| Tennesse | 3.83 |
| Arkansas | 3.83 |
| Utah | 3.86 |
| Kansas | 3.86 |
| Wyoming | 3.86 |
| North Ca | 3.88 |
| Virginia | 3.93 |
| Kentucky | 4.00 |
| Missouri | 4.00 |
| District | 4.03 |
| West Vir | 4.03 |
| Idaho | 4.03 |
| New Jers | 4.05 |
| Maryland | 4.05 |
| Delaware | 4.05 |
| Pennsylv | 4.08 |
| Nebraska | 4.08 |
| Iowa | 4.10 |
| Indiana | 4.10 |
| New York | 4.12 |
| Illinois | 4.15 |
| South Da | 4.15 |
| Montana | 4.15 |
| Rhode Is | 4.17 |
| Connecti | 4.17 |
| Ohio | 4.17 |
| Massachu | 4.20 |
| Wisconsi | 4.20 |
| Michigan | 4.20 |
| New Hamp | 4.22 |
| Minnesot | 4.22 |
| Maine | 4.25 |
| North Da | 4.25 |
| Oregon | 4.27 |
| Vermont | 4.29 |
| Washing | 4.34 |
| Alaska | 4.66 |

In Hawaii, there are 2.67 deaths and in Alaska, 4.66 deaths (per hundred thousand). 4.66/1=2.67/x. 4.66x=2.67. x=2.67/4.66. x=0.57296. In the place with the highest ultraviolet, deaths from melanoma are only about 57 percent the size. 1−0.57296=0.427. This is a predicted death rate from melanoma that is 42.7 percent lower or about 43 percent lower.

### Ovarian Cancer

Next is dying from ovarian cancer. Table 12-1 shows the state death rate from ovarian cancer in 2004-2006 (with the exception of Alaska being between the years 2003-2005).

TABLE 12-1
[ovar46ak] ovarian 2004-2006 Alaska. Ovarian Cancer
death rate by state between 2004 and 2006*, average annual,
per hundred thousand population (including both genders).

| | |
|---|---|
| Utah | 4.9 |
| Texas | 5.6 |
| Nevada | 5.7 |
| Colorado | 5.9 |
| Georgia | 6.0 |
| Hawaii | 6.2 |
| Californ | 6.3 |
| Idaho | 6.3 |
| Wyoming | 6.3 |
| Arizona | 6.5 |
| New Mexi | 6.5 |
| Louisian | 6.6 |
| Mississi | 6.8 |
| Arkansas | 6.9 |
| Kentucky | 7.0 |
| South Ca | 7.2 |
| New Hamp | 7.2 |
| North Ca | 7.3 |
| Virginia | 7.3 |
| Maryland | 7.3 |
| Illinois | 7.5 |
| Minnesot | 7.5 |
| Oklahoma | 7.6 |
| Kansas | 7.7 |
| Alaska | 7.7 |
| Washing | 7.7 |
| Missouri | 7.8 |
| Florida | 7.9 |
| Alabama | 7.9 |
| Michigan | 7.9 |
| Tennesse | 8.0 |
| Maine | 8.0 |
| Indiana | 8.0 |
| Ohio | 8.0 |
| Vermont | 8.0 |
| South Da | 8.0 |
| Delaware | 8.2 |
| Nebraska | 8.2 |
| Connecti | 8.3 |
| Wisconsi | 8.3 |
| New Jers | 8.4 |
| Oregon | 8.4 |
| New York | 8.5 |
| Massachu | 8.6 |
| Montana | 8.8 |
| West Vir | 8.9 |
| Iowa | 9.1 |
| North Da | 9.1 |
| District | 9.2 |
| Rhode Is | 9.3 |
| Pennsylv | 9.9 |

*Alaska from 2003-2005.

The ovarian model is in Table 12-4 consisting of two items: older age (more deaths) and ultraviolet in 1996 (stronger, fewer deaths).

TABLE 12-4
The Ovarian Cancer Model: the role of stronger
ultraviolet (fewer deaths).
Model Summary

| | | R | Adjusted R | Std. Error of the |
|---|---|---|---|---|
| Model | R | Square | Square | Estimate |
| 1 | .859(a) | .737 | .726 | .5603 |

(a)Predictors: (Constant), ULTRA96, AGE65IN6

Coefficients(a)

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | 4.259 | .639 | | 6.662 | .000 |
| | AGE65IN6 | .427 | .048 | .666 | 8.971 | .000 |
| | ULTRA96 | −.482 | .060 | −.600 | −8.080 | .000 |

(a)Dependent Variable: OVAR46AK

Correlations

| | | AGE65IN6 | ULTRA96 | OVAR46AK |
|---|---|---|---|---|
| AGE65IN6 | Pearson | 1 | .084 | .616(**) |
| | Correlation | | | |
| | Sig. | . | .560 | .000 |
| | (2-tailed) | | | |
| | N | 51 | 51 | 51 |
| ULTRA96 | Pearson | .084 | 1 | −.544(**) |
| | Correlation | | | |
| | Sig. | .560 | . | .000 |
| | (2-tailed) | | | |
| | N | 51 | 51 | 51 |
| OVAR46AK | Pearson | .616(**) | −.544(**) | 1 |
| | Correlation | | | |
| | Sig. | .000 | .000 | . |
| | (2-tailed) | | | |
| | N | 51 | 51 | 51 |

(**)Correlation is significant at the 0.01 level (2-tailed).

The predicted death rate list is in Table 12-5.

TABLE 12-5
[ovar461] ovarian 2004-2006 list. Best view predicted
death rate from ovarian cancer between 2004 and 2006 after
first largely neutralizing the effect of older age (more deaths)
in order to see the role of stronger ultraviolet (fewer deaths).
As only ultraviolet varies, this ordering of states is the
same as in Table 12-3 ultraviolet but reversed because stronger
ultraviolet, fewer deaths from ovarian cancer.

| | |
|---|---|
| Hawaii | 5.03 |
| Florida | 6.34 |
| Arizona | 6.67 |
| New Mexi | 6.72 |
| Texas | 6.82 |
| Californ | 6.82 |
| Louisian | 6.82 |
| Alabama | 6.87 |
| Nevada | 6.91 |
| Mississi | 7.01 |
| South Ca | 7.11 |
| Oklahoma | 7.16 |
| Georgia | 7.20 |
| Colorado | 7.30 |
| Arkansas | 7.35 |
| Tennesse | 7.35 |
| Utah | 7.40 |
| Wyoming | 7.40 |
| Kansas | 7.40 |
| North Ca | 7.44 |
| Virginia | 7.54 |
| Kentucky | 7.69 |
| Missouri | 7.69 |
| Idaho | 7.73 |
| West Vir | 7.73 |
| District | 7.73 |
| Maryland | 7.78 |
| Delaware | 7.78 |
| New Jers | 7.78 |
| Nebraska | 7.83 |
| Pennsylv | 7.83 |
| Indiana | 7.88 |
| Iowa | 7.88 |
| New York | 7.93 |
| Illinois | 7.97 |
| South Da | 7.97 |
| Montana | 7.97 |
| Ohio | 8.02 |
| Connecti | 8.02 |
| Rhode Is | 8.02 |
| Michigan | 8.07 |
| Wisconsi | 8.07 |
| Massachu | 8.07 |
| New Hamp | 8.12 |
| Minnesot | 8.12 |
| Maine | 8.17 |
| North Da | 8.17 |
| Oregon | 8.22 |
| Vermont | 8.26 |
| Washing | 8.36 |
| Alaska | 8.99 |

Holding older age constant, we can now see the size of the effect of ultraviolet on these death rates. In Hawaii, this is 5.03 deaths and in Alaska, it is 8.99 deaths. 8.99/1=5.03/x. 8.99x=5.03. x=5.0318.99. x=0.5595. Death rates from ovarian cancer are only about 56 percent the size in the state with the highest ultraviolet (compared with the lowest). 1−0.5595=0.4405. Due to ultraviolet, these predicted death rates from ovarian cancer are about 44 percent lower.

### Urinary Tract Cancer (Including Bladder)

Next is urinary tract cancer, a death category the majority of which is bladder cancer. The yearly death rate from urinary tract cancer by state is in Table 7-1 for the years 2004 and 2006.

TABLE 7-1
[urinry46] urinary tract cancer death rate, yearly average
between 2004 and 2006, majority from bladder cancer.

| | |
|---|---|
| Alaska | 4.3 |
| Utah | 5.2 |
| Hawaii | 5.8 |
| Georgia | 6.6 |
| Colorado | 6.9 |
| Californ | 7.2 |
| Texas | 7.3 |
| New Mexi | 7.7 |
| Virginia | 7.7 |
| Wyoming | 7.8 |
| Idaho | 7.9 |
| Mississi | 8.2 |
| Maryland | 8.2 |
| Arizona | 8.4 |
| South Ca | 8.4 |
| Alabama | 8.6 |
| Nevada | 8.6 |
| North Ca | 8.6 |
| New York | 8.7 |
| Illinois | 8.8 |
| Minnesot | 8.9 |
| Washing | 8.9 |
| Louisian | 9.2 |
| New Jers | 9.2 |
| Tennesse | 9.3 |
| Kansas | 9.3 |
| District | 9.3 |
| Michigan | 9.3 |
| New Hamp | 9.4 |
| Arkansas | 9.5 |
| Connecti | 9.6 |
| Montana | 9.7 |
| Oregon | 9.7 |
| Kentucky | 9.8 |
| Indiana | 9.8 |
| Nebraska | 10.0 |
| Oklahoma | 10.1 |
| Missouri | 10.1 |
| Massachu | 10.4 |
| Florida | 10.6 |
| Ohio | 10.7 |
| Wisconsi | 10.8 |
| Iowa | 11.0 |
| Delaware | 11.1 |
| West Vir | 11.4 |
| Pennsylv | 11.4 |
| Vermont | 11.4 |
| South Da | 11.8 |
| Rhode Is | 11.8 |
| North Da | 12.1 |
| Maine | 12.4 |

The model for urinary tract cancer appears in Table 7-4 and consists of two items.

TABLE 7-4
The model for urinary tract cancer: stronger ultraviolet, fewer deaths
Model Summary

| | | R | Adjusted R | Std. Error of the |
|---|---|---|---|---|
| Model | R | Square | Square | Estimate |
| 1 | .919(a) | .844 | .838 | .6978 |

(a)Predictors: (Constant), UVI0614, AGE65IN6

Coefficients(a)

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | .796 | .802 | | .993 | .325 |
| | AGE65IN6 | .874 | .059 | .844 | 14.755 | .000 |
| | UVI0614 | −.519 | .067 | −.446 | −7.790 | .000 |

(a)Dependent Variable: URINRY46

Correlations

| | | AGE65IN6 | UVI0614 | URINRY46 |
|---|---|---|---|---|
| AGE65IN6 | Pearson | 1 | .090 | .804(**) |
| | Correlation | | | |
| | Sig. | . | .531 | .000 |
| | (2-tailed) | | | |
| | N | 51 | 51 | 51 |
| UVI0614 | Pearson | .090 | 1 | −.370(**) |
| | Correlation | | | |
| | Sig. | .531 | . | .008 |
| | (2-tailed) | | | |
| | N | 51 | 51 | 51 |
| URINRY46 | Pearson | .804(**) | −.370(**) | 1 |
| | Correlation | | | |
| | Sig. | .000 | .008 | . |
| | (2-tailed) | | | |
| | N | 51 | 51 | 51 |

(**)Correlation is significant at the 0.01 level (2-tailed).

More people in a state who are in the age 65 and over category, more deaths. The second item is the intensity of ultraviolet reaching the ground over the course of the year in the largest city of the state. This is for the years 2006 to 2014. The stronger the ultraviolet, the fewer the deaths from cancers of the urinary tract.

The reworked predicted death rates appear in Table 7-5.

TABLE 7-5
“Best view” predicted death rates from cancer of the
urinary tract including bladder. Based on actual death rates
in 2004-2006. The effect of older age (more deaths) is largely
removed in order to see the size of the impact of more intense
ultraviolet (fewer deaths). As only ultraviolet varies, the
state order is the same as in Table 7-3 ultraviolet but in reverse.

| | |
|---|---|
| Hawaii | 6.1 |
| Florida | 7.4 |
| Louisian | 8.1 |
| Californ | 8.2 |
| New Mexi | 8.2 |
| Alabama | 8.2 |
| Texas | 8.3 |
| Arizona | 8.3 |
| Mississi | 8.5 |
| South Ca | 8.6 |
| Nevada | 8.6 |
| Georgia | 8.7 |
| Colorado | 8.8 |
| Utah | 8.9 |
| Arkansas | 8.9 |
| Oklahoma | 8.9 |
| Wyoming | 9.0 |
| Tennesse | 9.0 |
| Virginia | 9.1 |
| North Ca | 9.1 |
| Kansas | 9.2 |
| Idaho | 9.3 |
| Kentucky | 9.4 |
| Missouri | 9.4 |
| Maryland | 9.5 |
| New Jers | 9.5 |
| District | 9.5 |
| Delaware | 9.5 |
| West Vir | 9.5 |
| Indiana | 9.5 |
| Nebraska | 9.5 |
| New York | 9.6 |
| Montana | 9.6 |
| Pennsylv | 9.6 |
| Iowa | 9.6 |
| Illinois | 9.7 |
| Connecti | 9.7 |
| Massachu | 9.7 |
| Ohio | 9.7 |
| South Da | 9.7 |
| Rhode Is | 9.7 |
| Wisconsi | 9.7 |
| Michigan | 9.8 |
| New Hamp | 9.8 |
| Minnesot | 9.8 |
| North Da | 9.8 |
| Maine | 9.8 |
| Oregon | 10.0 |
| Vermont | 10.0 |
| Washing | 10.0 |
| Alaska | 10.9 |

Older age is held constant so we can see the size of the effect of ultraviolet by itself on these death rates. In Hawaii, this urinary tract death rate is 6.1 deaths per hundred thousand people. In Alaska, it is 10.9 deaths. 10.9/1=6.1/x. 10.9x=6.1. x=6.1/10.9. x=0.5596. Due to stronger ultraviolet, the death rate from urinary tract cancer is only 0.5596 or about 56 percent the size. 1−0.55963=0.44037. This predicted death rate from urinary tract cancer is 44 percent lower due to stronger ultraviolet.

### Uterine Cancer

Next is uterine cancer. The death rate for uterine cancer by state can be found in Table 13-1 for the years 2012-2014.

TABLE 13-1
[uter1214] uterine 2012-2014. Average annual death
rate from uterine cancer between 2012 and 2014 by state
(per hundred thousand population including both women and men).

| | |
|---|---|
| Alaska | 2.2 |
| Nevada | 2.5 |
| Utah | 2.5 |
| Colorado | 2.7 |
| Wyoming | 2.8 |
| Texas | 2.9 |
| North Da | 2.9 |
| Alabama | 3.0 |
| Arizona | 3.1 |
| Oklahoma | 3.1 |
| Arkansas | 3.1 |
| Hawaii | 3.2 |
| New Mexi | 3.2 |
| Mississi | 3.2 |
| Californ | 3.4 |
| Louisian | 3.4 |
| Tennesse | 3.4 |
| Idaho | 3.4 |
| Georgia | 3.5 |
| Kentucky | 3.5 |
| Minnesot | 3.5 |
| Washing | 3.5 |
| South Da | 3.6 |
| Kansas | 3.7 |
| Virginia | 3.7 |
| Nebraska | 3.8 |
| Montana | 3.8 |
| South Ca | 3.9 |
| North Ca | 3.9 |
| Missouri | 3.9 |
| Connecti | 3.9 |
| New Hamp | 3.9 |
| Oregon | 3.9 |
| Florida | 4.0 |
| Massachu | 4.0 |
| Wisconsi | 4.1 |
| Indiana | 4.3 |
| Illinois | 4.4 |
| Ohio | 4.4 |
| Michigan | 4.4 |
| Maine | 4.4 |
| West Vir | 4.5 |
| Rhode Is | 4.6 |
| Maryland | 4.7 |
| Vermont | 4.8 |
| Iowa | 4.9 |
| New York | 4.9 |
| New Jers | 5.0 |
| Pennsylv | 5.1 |
| District | 5.2 |
| Delaware | 5.3 |

The model for uterine is in Table 13-5.

TABLE 13-5
The Uterine Cancer Model: the role of particle air pollution (more deaths)
as well as stronger ultraviolet in sunlight (fewer deaths).
Model Summary

| | | R | Adjusted R | Std. Error of the |
|---|---|---|---|---|
| Model | R | Square | Square | Estimate |
| 1 | .766(a) | .587 | .560 | .5018 |

(a)Predictors: (Constant), ULTRA96, PMMETRO, AGE65IN6

Coefficients(a)

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | .433 | .633 | | .684 | .497 |
| | AGE65IN6 | .247 | .043 | .546 | 5.800 | .000 |
| | PMMETRO | 8.824E−02 | .020 | .424 | 4.516 | .000 |
| | ULTRA96 | −.211 | .054 | −.371 | −3.944 | .000 |

(a)Dependent Variable: UTER1214

Correlations

| | | AGE65IN6 | PMMETRO | ULTRA96 | UTER1214 |
|---|---|---|---|---|---|
| AGE65IN6 | Pearson | 1 | −.018 | .084 | .507(**) |
| | Correlation | | | | |
| | Sig. | . | .899 | .560 | .000 |
| | (2-tailed) | | | | |
| | N | 51 | 51 | 51 | 51 |
| PMMETRO | Pearson | −.018 | 1 | −.042 | .430(**) |
| | Correlation | | | | |
| | Sig. | .899 | . | .769 | .002 |
| | (2-tailed) | | | | |
| | N | 51 | 51 | 51 | 51 |
| ULTRA96 | Pearson | .084 | −.042 | 1 | −.344(*) |
| | Correlation | | | | |
| | Sig. | .560 | .769 | . | .014 |
| | (2-tailed) | | | | |
| | N | 51 | 51 | 51 | 51 |
| UTER1214 | Pearson | .507(**) | .430(**) | −.344(*) | 1 |
| | Correlation | | | | |
| | Sig. | .000 | .002 | .014 | . |
| | (2-tailed) | | | | |
| | N | 51 | 51 | 51 | 51 |

(**)Correlation is significant at the 0.01 level (2-tailed).
(*)Correlation is significant at the 0.05 level (2-tailed).

It has three items. Older age, more deaths. Particle air pollution in the metropolitan area of the largest city of the state, more deaths. Lastly is ultraviolet, the yearly average for 1996 in the largest city. The more intense this ultraviolet reaching the ground, the fewer the deaths from uterine cancer (for the size of the population).

In the book, I chose in Table 13-6 (not shown) to hold constant only one of the two items so the predicted rate there does not give us what we need right now—the specific effect of ultraviolet on these death rates. For this reason, I redid the exercise for this purpose in a supplemental table I will call Table 13-6-2.

TABLE 13-6-2
[uteruv13] uterine uv list 3. Supplemental table showing
the specific size of the ultraviolet effect on dying from
uterine cancer. Here, both older age and particle air pollution
are held constant and only ultraviolet is allowed to vary.

| | |
|---|---|
| Hawaii | 2.67 |
| Florida | 3.24 |
| Arizona | 3.39 |
| New Mexi | 3.41 |
| Louisian | 3.45 |
| Californ | 3.45 |
| Texas | 3.45 |
| Alabama | 3.47 |
| Nevada | 3.49 |
| Mississi | 3.54 |
| South Ca | 3.58 |
| Oklahoma | 3.60 |
| Georgia | 3.62 |
| Colorado | 3.66 |
| Arkansas | 3.68 |
| Tennesse | 3.68 |
| Utah | 3.70 |
| Wyoming | 3.70 |
| Kansas | 3.70 |
| North Ca | 3.73 |
| Virginia | 3.77 |
| Kentucky | 3.83 |
| Missouri | 3.83 |
| Idaho | 3.85 |
| West Vir | 3.85 |
| District | 3.85 |
| Delaware | 3.87 |
| New Jers | 3.87 |
| Maryland | 3.87 |
| Nebraska | 3.89 |
| Pennsylv | 3.89 |
| Indiana | 3.92 |
| Iowa | 3.92 |
| New York | 3.94 |
| Montana | 3.96 |
| Illinois | 3.96 |
| South Da | 3.96 |
| Ohio | 3.98 |
| Connecti | 3.98 |
| Rhode Is | 3.98 |
| Massachu | 4.00 |
| Wisconsi | 4.00 |
| Michigan | 4.00 |
| New Hamp | 4.02 |
| Minnesot | 4.02 |
| Maine | 4.04 |
| North Da | 4.04 |
| Oregon | 4.06 |
| Vermont | 4.08 |
| Washing | 4.13 |
| Alaska | 4.40 |

In this recalculation, the predicted death rate in Alaska is 4.40 deaths while in Hawaii, it is only 2.67 deaths. 4.40/1=2.67/x. 4.40x=2.67. x=2.6714.40. x=0.6068. Due to ultraviolet, the death rate is only about 61 percent the size in Hawaii compared with Alaska. 1−0.6068=0.3932. The death rate from uterine cancer, due to stronger ultraviolet, is lower by 39.32 percent or by around 39 percent.

### Lymphoma, Leukemia and Prostate

Now we are at the second group of three cancers including lymphoma, adult leukemia and prostate. In the book, I used models suggesting a different environmental factor which varies as we drive north might be responsible for this pattern of more deaths. For the purpose here, I rather easily took each of the three models and replaced this item (geomagnetism) with ultraviolet. From the new models that resulted, I used the equation to create a new predicted death rate list for each cancer so we could see the size of the effect of ultraviolet in shrinking these death rates as well.

To repeat the rationale, the exercise is based on the reasonable possibility that only one environmental factor might be responsible for the northern pattern and that it might, in the end, be the difference in the intensity of sunlight between places. Of course, the ultraviolet portion of sunlight gets weaker as we go north in the United States.

### Non-Hodgkin's Lymphoma

The first cancer in this group is Non-Hodgkin's lymphoma. The starting death rate is unchanged and can be found in Table 8-1 for the years 2004-2006.

TABLE 8-1
Lymphoma: Non-Hodgkin's lymphoma
death rates by state 2004-2006

| | |
|---|---|
| Alaska | 3.5 |
| Utah | 5.2 |
| Colorado | 5.2 |
| Georgia | 5.3 |
| Nevada | 5.4 |
| New Mexi | 5.4 |
| Texas | 5.6 |
| Californ | 5.8 |
| Hawaii | 5.9 |
| Mississi | 5.9 |
| Virginia | 6.1 |
| Maryland | 6.2 |
| New Hamp | 6.3 |
| Arizona | 6.5 |
| South Ca | 6.5 |
| Idaho | 6.6 |
| North Ca | 6.8 |
| Vermont | 6.8 |
| New York | 6.9 |
| Wyoming | 6.9 |
| Louisian | 7.1 |
| Alabama | 7.1 |
| Delaware | 7.1 |
| Washing | 7.1 |
| Illinois | 7.1 |
| New Jers | 7.3 |
| Oklahoma | 7.4 |
| District | 7.5 |
| Missouri | 7.5 |
| Arkansas | 7.6 |
| Kansas | 7.6 |
| Connecti | 7.6 |
| Minnesot | 7.6 |
| Kentucky | 7.7 |
| Indiana | 7.8 |
| Ohio | 7.8 |
| Michigan | 7.8 |
| Tennesse | 7.9 |
| Rhode Is | 7.9 |
| Montana | 7.9 |
| Nebraska | 8.1 |
| Oregon | 8.2 |
| Massachu | 8.2 |
| Wisconsi | 8.2 |
| South Da | 8.2 |
| Maine | 8.5 |
| Florida | 8.6 |
| Iowa | 8.7 |
| North Da | 8.9 |
| West Vir | 9.3 |
| Pennsylv | 9.4 |

Using the book model in Table 8-4 (not shown), I will now replace the environmental item with ultraviolet in Table 8-4-2.

TABLE 8-4-2
Lymphoma Model with Ultraviolet (fewer deaths)
Model Summary

| | | R | Adjusted R | Std. Error of the |
|---|---|---|---|---|
| Model | R | Square | Square | Estimate |
| 1 | .912^{a} | .832 | .824 | .4987 |

^{a}Predictors: (Constant), UVI0614, ACE65IN6

Coefficients^{a}

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | .699 | .573 | | 1.219 | .229 |
| | ACE65IN6 | .622 | .042 | .874 | 14.688 | .000 |
| | UVI0614 | −.281 | .048 | −.351 | −5.896 | .000 |

^{a}Dependent Variable: LYMPH46

Correlations

| | | ACE65IN6 | UVI0614 |
|---|---|---|---|
| ACE65IN6 | Pearson | 1 | .090 |
| | Correlation | | |
| | Sig. | . | .531 |
| | (2-tailed) | | |
| | N | 51 | 51 |
| UVI0614 | Pearson | .090 | 1 |
| | Correlation | | |
| | Sig. | .531 | . |
| | (2-tailed) | | |
| | N | 51 | 51 |

The adjusted R Square is 0.824, slightly below the 0.837 of the original model. It includes the more recent ultraviolet measure for the years 2006-2014.

Using the equation in this model, I hold constant the age effect to see the size of the ultraviolet one which is the only thing to vary. According to this predicted list of death rates from lymphoma in Table 8-5-2,

TABLE 8-5-2
[lymphuvl]. Predicted deaths from lymphoma after accounting
for older age based on the effect of differences in the intensity
of ultraviolet reaching the ground during the yuears 2006-2014.

| | |
|---|---|
| Hawaii | 5.45 |
| Florida | 6.18 |
| Louisian | 6.55 |
| New Mexi | 6.60 |
| Californ | 6.60 |
| Alabama | 6.60 |
| Arizona | 6.63 |
| Texas | 6.63 |
| Mississi | 6.77 |
| Nevada | 6.80 |
| South Ca | 6.80 |
| Georgia | 6.88 |
| Colorado | 6.94 |
| Utah | 6.97 |
| Oklahoma | 7.00 |
| Arkansas | 7.00 |
| Tennesse | 7.02 |
| Wyoming | 7.02 |
| North Ca | 7.05 |
| Virginia | 7.05 |
| Kansas | 7.11 |
| Idaho | 7.19 |
| Kentucky | 7.22 |
| Missouri | 7.22 |
| West Vir | 7.28 |
| District | 7.28 |
| Delaware | 7.28 |
| New Jers | 7.28 |
| Maryland | 7.28 |
| Nebraska | 7.30 |
| Indiana | 7.30 |
| Pennsylv | 7.33 |
| New York | 7.33 |
| Montana | 7.33 |
| Iowa | 7.36 |
| Illinois | 7.39 |
| South Da | 7.39 |
| Ohio | 7.39 |
| Connecti | 7.39 |
| Rhode Is | 7.39 |
| Massachu | 7.39 |
| Wisconsi | 7.42 |
| Michigan | 7.45 |
| New Hamp | 7.45 |
| Minnesot | 7.47 |
| Maine | 7.47 |
| North Da | 7.47 |
| Oregon | 7.56 |
| Vermont | 7.56 |
| Washing | 7.59 |
| Alaska | 8.04 |

The highest is for Alaska with 8.04 deaths per hundred thousand. The lowest is Hawaii with 5.45 deaths. 8.04/1=5.45/x. 5.45=8.04x. x=5.45/8.04. x=0.67786. Based on the difference in ultraviolet alone, the death rate from lymphoma in the lowest death rate state of Hawaii is only 0.67786 or about 68 percent the size. 1−0.67786=0.32214. Due to stronger ultraviolet, the predicted death rate from lymphoma declines by 32.2 percent or by close to a third.

### Adult Leukemia

Second now in this group of three cancers is leukemia. This does not include deaths among children as all the analyses are for people age 18 and older. We start with the death rates for this cancer between the years 2011-2013 in Table 10-1.

TABLE 10-1
Adult Leukemia. Death rates from leukemia in 2011-2013 by state.

| | |
|---|---|
| Alaska | 5.3 |
| District | 6.4 |
| Utah | 6.5 |
| Hawaii | 7.4 |
| Georgia | 7.5 |
| Colorado | 7.5 |
| Texas | 7.9 |
| New Mexi | 8.0 |
| Californ | 8.1 |
| Nevada | 8.2 |
| Virginia | 8.3 |
| Maryland | 8.8 |
| Washing | 8.9 |
| Idaho | 8.9 |
| North Da | 9.0 |
| Vermont | 9.1 |
| North Ca | 9.1 |
| New York | 9.2 |
| Louisian | 9.2 |
| New Jers | 9.3 |
| South Ca | 9.3 |
| Delaware | 9.5 |
| Arizona | 9.5 |
| Massachu | 9.8 |
| New Hamp | 9.8 |
| Illinois | 9.9 |
| Mississi | 9.9 |
| Montana | 9.9 |
| Oregon | 10.1 |
| Michigan | 10.2 |
| Rhode Is | 10.3 |
| Tennesse | 10.3 |
| Connecti | 10.4 |
| Kentucky | 10.5 |
| Nebraska | 10.6 |
| Missouri | 10.6 |
| Minnesot | 10.7 |
| Indiana | 10.7 |
| Ohio | 10.8 |
| Alabama | 10.8 |
| Florida | 10.9 |
| Kansas | 11.0 |
| Arkansas | 11.0 |
| Oklahoma | 11.0 |
| Wyoming | 11.1 |
| Maine | 11.3 |
| Wisconsi | 11.4 |
| Pennsylv | 11.5 |
| Iowa | 11.6 |
| South Da | 11.9 |
| West Vir | 12.5 |

Using the leukemia model in Table 10-5 (not shown), we rework it replacing magnetism with ultraviolet in Table 10-5-2.

TABLE 10-5-2
Leukemia Model with Ultraviolet (fewer deaths)
Model Summary

| | | R | Adjusted R | Std. Error of the |
|---|---|---|---|---|
| Model | R | Square | Square | Estimate |
| 1 | .876^{a} | .767 | .752 | .7525 |

^{a}Predictors: (Constant), IVI0614, AGE65IN6, DOGHOUSE

Coefficients^{a}

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | −1.052 | 1.049 | | −1.002 | .321 |
| | ACE65IN6 | .772 | .065 | .854 | 11.810 | .000 |
| | DOGHOUSE | 8.978E−02 | .017 | .404 | 5.342 | .000 |
| | UVI0614 | −.388 | .076 | −.382 | −5.117 | .000 |

^{a}Dependent Variable: LEUK1113

Correlations

| | | AGE65IN6 | DOGHOUSE | UVI0614 | LEUK1113 |
|---|---|---|---|---|---|
| AGE65IN6 | Pearson | 1 | −.175 | .090 | .750* |
| | Correlation | | | | |
| | Sig. | . | .220 | .531 | .000 |
| | (2-tailed) | | | | |
| | N | 51 | 51 | 51 | 51 |
| DOGHOUSE | Pearson | −.175 | 1 | .300* | .139 |
| | Correlation | | | | |
| | Sig. | .220 | . | .032 | .329 |
| | (2-tailed) | | | | |
| | N | 51 | 51 | 51 | 51 |
| UVI0614 | Pearson | .090 | .300* | 1 | −.184 |
| | Correlation | | | | |
| | Sig. | .531 | .032 | . | .196 |
| | (2-tailed) | | | | |
| | N | 51 | 51 | 51 | 51 |
| LEUK1113 | Pearson | .750* | .139 | −.184 | 1 |
| | Correlation | | | | |
| | Sig. | .000 | .329 | .196 | . |
| | (2-tailed) | | | | |
| | N | 51 | 51 | 51 | 51 |

**Correlation is significant at the 0.01 level (2-tailed).
*Correlation is significant at the 0.05 level (2-tailed).

To my surprise, the Adjusted R Square comes out the same as the original model, 0.752 (something I was surely not aware of at the time I was writing the book).

Using the equation in this ultraviolet model, I hold constant both older age (more deaths) and having a dog (more deaths) to see the specific effect of ultraviolet (more ultraviolet, fewer deaths) in Table 10-6-2.

TABLE 10-6-2
[leukuvil] Leukemia UV Index List. Predicted death rate
from Leukemia after accounting for older age and having a
dog to see the size of the ultraviolet effect (fewer deaths)

| | |
|---|---|
| Hawaii | 7.32 |
| Florida | 8.33 |
| Louisian | 8.83 |
| New Mexi | 8.91 |
| Californ | 8.91 |
| Alabama | 8.91 |
| Arizona | 8.95 |
| Texas | 8.95 |
| Mississi | 9.14 |
| Nevada | 9.18 |
| South Ca | 9.18 |
| Georgia | 9.30 |
| Colorado | 9.38 |
| Utah | 9.41 |
| Oklahoma | 9.45 |
| Arkansas | 9.45 |
| Tennesse | 9.49 |
| Wyoming | 9.49 |
| North Ca | 9.53 |
| Virginia | 9.53 |
| Kansas | 9.61 |
| Idaho | 9.72 |
| Kentucky | 9.76 |
| Missouri | 9.76 |
| West Vir | 9.84 |
| District | 9.84 |
| Delaware | 9.84 |
| New Jers | 9.84 |
| Maryland | 9.84 |
| Nebraska | 9.88 |
| Indiana | 9.88 |
| Pennsylv | 9.92 |
| New York | 9.92 |
| Montana | 9.92 |
| Iowa | 9.96 |
| Illinois | 10.00 |
| South Da | 10.00 |
| Ohio | 10.00 |
| Connecti | 10.00 |
| Rhode Is | 10.00 |
| Massachu | 10.00 |
| Wisconsi | 10.03 |
| Michigan | 10.07 |
| New Hamp | 10.07 |
| Minnesot | 10.11 |
| Maine | 10.11 |
| North Da | 10.11 |
| Oregon | 10.23 |
| Vermont | 10.23 |
| Washing | 10.27 |
| Alaska | 10.89 |

The ultraviolet item that works best here is for the nine years between 2006 and 2014. The predicted death rate from leukemia is highest in Alaska at 10.89 deaths and lowest in Hawaii at 7.32 deaths. This difference can be accounted for entirely due to the effect of ultraviolet on these death rates, based on this model. 10.89/1=7.32/x. 10.89x=7.32. x=7.32/10.89. x=0.67218. Due to stronger ultraviolet, the predicted death rate in Hawaii from leukemia is only some two thirds the size that it is in the state with the highest death rate (some 67 percent). 1−0.67218=0.328. This means that the death rate from adult leukemia is reduced by 32.8 percent, almost 33 percent or by almost a third.

### Prostate Cancer

Lastly now is prostate cancer. Again here, we begin with the actual death rate list for prostate cancer deaths between 2010 and 2012 which can be found in Table 11-1.

TABLE 11-1
[pros1012] Death rate from prostate cancer in 2010-
2012 per hundred thousand people (including women).

| | |
|---|---|
| Alaska | 7.3 |
| Texas | 8.8 |
| Hawaii | 9.4 |
| Georgia | 10.4 |
| Utah | 10.7 |
| Colorodo | 10.7 |
| Californ | 10.7 |
| Nevada | 10.8 |
| Virginia | 10.9 |
| Kentucky | 10.9 |
| Maryland | 11.0 |
| Mississi | 11.1 |
| Wyoming | 11.4 |
| New York | 11.5 |
| Washing | 11.6 |
| New Jers | 11.7 |
| Kansas | 11.7 |
| Illinois | 11.7 |
| Massachu | 11.7 |
| Arizona | 11.8 |
| Tennesse | 11.8 |
| North Ca | 11.9 |
| New Hamp | 12.0 |
| Connecti | 12.0 |
| Indiana | 12.1 |
| Michigan | 12.1 |
| Louisian | 12.1 |
| West Vir | 12.1 |
| New Mexi | 12.5 |
| Oklahoma | 12.5 |
| Delaware | 12.6 |
| Minnesot | 12.6 |
| Rhode Is | 12.7 |
| Ohio | 12.7 |
| Maine | 13.0 |
| Nebraska | 13.2 |
| South Ca | 13.2 |
| Arkansas | 13.3 |
| Pennsylv | 13.5 |
| Iowa | 13.6 |
| Idaho | 13.7 |
| North Oa | 13.8 |
| Wisconsi | 13.8 |
| Oregon | 13.8 |
| Fiorida | 13.8 |
| South Da | 14.1 |
| Alabama | 14.1 |
| Vermont | 14.1 |
| Montana | 14.7 |
| Missouri | 14.8 |
| District | 14.8 |

Taking the prostate model from the book in Table 11-4 (not shown), we change geomagnetism to our relevant item that varies as we go north, ultraviolet, in the new model in Table 11-4-2.

TABLE 11-4-2
Prostate Model with Ultraviolet
Model Summary

| | | R | Adjusted R | Std. Error of the |
|---|---|---|---|---|
| Model | R | Square | Square | Estimate |
| 1 | .728^{a} | .530 | .510 | 1.0657 |

^{a}Predictors: (Constant), ULTRA96, ACE65IN6

Coefficients^{a}

| | | Unstandardized | | | | |
|---|---|---|---|---|---|---|
| | | Coefficients | | Standardized | | |
| | | | Std. | Coefficients | | |
| Model | | B | Error | Beta | t | Sig. |
| 1 | (Constant) | 5.476 | 1.216 | | 4.503 | .000 |
| | AGE65IN6 | .637 | .090 | .700 | 7.045 | .000 |
| | ULTRA96 | −.307 | .114 | −.268 | −2.702 | .009 |

^{a}Dependent Variable: PROS1012

Correlations

| | | AGE65IN6 | ULTRA96 | PROS1012 |
|---|---|---|---|---|
| ACE65IN6 | Pearson | 1 | .084 | .677* |
| | Correlation | | | |
| | Sig. | . | .560 | .000 |
| | (2-tailed) | | | |
| | N | 51 | 51 | 51 |
| ULTRA96 | Pearson | .084 | 1 | −.210 |
| | Correlation | | | |
| | Sig. | .560 | . | .139 |
| | (2-tailed) | | | |
| | N | 51 | 51 | 51 |
| PROS1012 | Pearson | .677* | −.210 | 1 |
| | Correlation | | | |
| | Sig. | .000 | .139 | . |
| | (2-tailed) | | | |
| | N | 51 | 51 | 51 |

** Correlation is significant at the 0.01 level (2-tailed).

In this original model, the Adjusted R Square is 0.583. In this replacement model, the Adjusted R Square is 0.510, not as high. As mentioned, the statistical significance is 0.009, not as good as other models but still, below the 0.01 limit I use and well below the 0.05 limit that is more common.

Holding constant the effect of older age now, we can see the size of the intensity of ultraviolet on prostate cancer death rates in Table 11-5-2.

TABLE 11-5-2
[prosuvl2]. Prostate UV List 2. Predicted death rate
from prostate cancer holding older age constant to see the
size of the effect of ultraviolet on deaths from this cancer.

| | |
|---|---|
| Hawaii | 10.59 |
| Florida | 11.42 |
| Arizona | 11.63 |
| New Mexi | 11.66 |
| Louisian | 11.72 |
| Californ | 11.72 |
| Texas | 11.72 |
| Alabama | 11.76 |
| Nevada | 11.79 |
| Mississi | 11.85 |
| South Ca | 11.91 |
| Oklahoma | 11.94 |
| Georgia | 11.97 |
| Colorado | 12.03 |
| Arkansas | 12.06 |
| Tennesse | 12.06 |
| Utah | 12.09 |
| Wyoming | 12.09 |
| Kansas | 12.09 |
| North Ca | 12.12 |
| Virginia | 12.19 |
| Kentucky | 12.28 |
| Missouri | 12.28 |
| Idaho | 12.31 |
| West Vir | 12.31 |
| District | 12.31 |
| Delaware | 12.34 |
| New Jers | 12.34 |
| Maryland | 12.34 |
| Nebraska | 12.37 |
| Pennsylv | 12.37 |
| Indiana | 12.40 |
| Iowa | 12.40 |
| New York | 12.43 |
| Montana | 12.46 |
| Illinois | 12.46 |
| South Da | 12.46 |
| Ohio | 12.49 |
| Connecti | 12.49 |
| Rhode Is | 12.49 |
| Massachu | 12.52 |
| Wisconsi | 12.52 |
| Michigan | 12.52 |
| New Hamp | 12.55 |
| Minnesot | 12.55 |
| Maine | 12.58 |
| North Da | 12.58 |
| Oregon | 12.61 |
| Vermont | 12.65 |
| Washing | 12.71 |
| Alaska | 13.11 |

In this model, ultraviolet in 1996 worked best. For prostate cancer, the predicted death rate goes from a high of 13.11 deaths in Alaska to a low of 10.59 deaths in Hawaii. 13.11/1=10.59/x. 13.11x=10.59. x=10.59/13.11. x=0.80778. Due to difference in the intensity of ultraviolet, the death rate from prostate cancer in the place with the strongest “sun” is only some 80.8 percent. 1−0.80778=0.192. This is a death rate that is 19.2 percent lower.

While these numbers are difficult to translate into how effective an ultraviolet-based device might be for treating these eight northern cancers, they probably give us some indication of the effectiveness that might be expected.

We can now summarize the results in size order based on this percent decrease from the state with the lowest intensity of ultraviolet, Alaska, to the one with the highest level, Hawaii. Here is the percent decrease in death rates for the eight northern cancers in size order that can be attributed specifically to the difference in the intensity of ultraviolet in sunlight reaching the ground over the course of the year. The more intense the sunlight, the fewer the deaths.

1. ovarian—44.1 percent lower
2. urinary (including bladder)—44.0 percent lower
3. melanoma—41.7 percent lower
4. uterine—39.3 percent lower
5. leukemia (adult)—32.8 percent lower
6. lymphoma (Non-Hodgkin's)—32.2 percent lower
7. lung—26.1 percent lower
8. prostate—19.2 percent lower

### Keith Reed Greenbaum, Ph.D.

### 284 Brook Street Apartment 5

### Providence, R.I. 02906

### United States of America

Email: keithmeister@hotmail.com

1-34. (canceled)

35. The invention is a device for treating cancer in eight death categories with ultraviolet radiation including lung, melanoma, ovarian, urinary, uterine, non-Hodgkin's lymphoma, adult leukemia and prostate.

36. The said device in claim 35 is importantly defined by its two intensity values, its total intensity of between 6.6 to around 9.6 as a UV Index score, and the intensity value in the UVB range of 3 watts per square meter.

37. The origin of the intensity values in claim 36 are as follows: 9.6 from the statistical research, the yearly UV Index value for Hawaii (in one of two versions), 6.6 an average measured value in Honolulu between summer and winter, to simulate the value over the year, 3 watts per square meter UVB from several summer and winter values in Honolulu, 3.4 and 2.4, to average 2.9, rounded to 3.

38. The said device in claim 35 includes ultraviolet in both ranges which naturally reach the ground: UVB and UVA.

39. The said device in claim 35 includes wavelengths from 280 to 400 nanometers.

40. The said device in claim 35 consists of rows of florescent tubes emitting ultraviolet radiation, four in the small and medium versions and 6 in the large version.

41. The dimensions for the versions in claim 40 are as follows: small—22 inches wide, 9 inches deep and 14 inches high; medium—2 feet tall, one foot wide; large—to hold one row of five foot bulbs in a frame that can be tilted for use standing or lying.

42. The intensity values in claim 36 are achieved by positioning the said device in claim 35 the appropriate distance from the face or the area of the cancer using the two appropriate solarmeter.com meters, the first a UV Index meter Model 6.5, and the second a UVB meter Model 6.0 (after converting milliwatts per centimeter square to watts per meter square).

43. The foundation for the device in claim 35 is a statistical connection in a “big data” project linking the death rate, of such cancers by state in the United States to the insufficiently strong intensity of ultraviolet reaching the ground in sunlight in that state over the course of the year.

44. The said connection in claim 42 is between two sets of numbers, the death rate from the various cancers and a special yearly rendering of the UV Index that captures the intensity of ultraviolet reaching in the ground in the largest city of the state in two versions, one for the year 1996 and the other for a nine year period between 2006 and 2014.

45. The said connection in claim 44 shows that the lower the UV Index over the year in a place, the higher the cancer death rate in that place after accounting for the other items in the model.

46. The evidence presented in claim 45 improves over the prior art by strengthening the connection between cancer death rates and a specific measure of ultraviolet intensity, across more data points—the 51 US states (including Washington D.C.), in urban areas using the largest city to represent the state, and including all groups of the American population.

47. The said invention in claim 35 is designed for exclusively treating cancer that has already taken hold and is not intended to be used for cancer prevention.

48. The said invention in claim 35 is unconnected with a cancer prevention strategy of vitamin D (and calcium) supplements which have shown no overall success in preventing cancer as of 2019 in a prospective study.

49. Treatment with ultraviolet as proposed here in claim 35 is largely separate from research on vitamin D in the blood 25(OH)D which includes the impact of vitamin D supplements which might not have the same effect as ultraviolet itself.

50. While the application here does not look at “inside the body” mechanisms, the chance exists that ultraviolet as proposed here in claim 35 might work entirely independently from vitamin D in an, as yet, unspecified way to fight cancer once it has taken hold.

51. After showing a statistical connection with ultraviolet for each of the cancers in eight death categories, this evidence foundation suggests the proposed ultraviolet device (with the above specifications) might treat these cancers which include lung, melanoma, ovarian, urinary, uterine, non-Hodgkin's lymphoma, adult leukemia and prostate.
